(19) United States
(12) Patent Application Publication
(10) Pub. No.: US 2023/0028920 A1
(43) Pub. Date: Jan. 26, 2023

(54) DETECTION OF SEIZURE EVENTS

(71) Applicant: Hewlett-Packard Development Company, L.P., Spring, TX (US)

(72) Inventor: Christopher Steven, Spring, TX (US)

(21) Appl. No.: 17/778,343

(22) PCT Filed: Nov. 20, 2019
(86) PCT No.: PCT/US2019/062328

§ 371 Date: May 19, 2022

(51) Int. Cl. A61B 5/00; A61B 5/11

(52) U.S. Cl. CPC A61B 5/4094; A61B 5/1114; A61B 5/746

(57) ABSTRACT

In some examples, a device for detecting seizure events can include a non-transitory machine readable medium storing instructions executable by a processing resource to generate a point cloud of an object, monitor a movement of the generated point cloud based on data received from a sensor, and detect a seizure event based on the monitored movement

### BACKGROUND

It is estimated that 50 million people have epilepsy worldwide. A seizure can generally happen anytime, anywhere, and very often without predictive signs, which can be especially dangerous when the patient is alone. For patients with photosensitive epilepsy (PSE), seizures can be triggered by visual stimuli, such as flashing lights, regular still or moving patterns. Computing devices have traditionally been a common source of seizures in PSE patients, for example, when the computing device is out of adjustment or is showing a rapidly flickering image.

### BRIEF DESCRIPTION OF THE DRAWINGS

FIG. 1 is an example of a point cloud for the detection of seizure events consistent with the disclosure.

FIG. 2 is an example of a system for the detection of seizure events consistent with the disclosure.

FIG. 3 illustrates an example of a memory resource storing instructions for the detection of seizure events consistent with the disclosure.

FIG. 4 illustrates an example of a computing device for the detection seizure events consistent with the disclosure.

FIG. 5 is an example of a method for the detection of seizure events consistent with the disclosure.

### DETAILED DESCRIPTION

As used herein, the term “computing device” can be, for example, a laptop computer, a desktop computer, or a mobile device (e.g., a smart phone, tablet, personal digital assistant, smart glasses, a wrist-worn device, etc.), among other types of computing devices. As used herein, the term “mobile device” refers to a device that are (or can be) carried and/or worn by a user. A mobile device can include a phone (e.g., a smart phone), a tablet, a personal digital assistant (PDA), smart glasses, and/or a wrist-worn device (e.g., a smart watch), among other types of mobile devices.

As used herein, the term “sensor” refers to a device to detect events and/or changes in its environment and transmit the detected events and/or changes for processing and/or analysis. For example, the sensor can detect events or changes, such as a movement of a user. Sensors can include a virtual accelerometer, ultrasonic detector, passive infrared motion detector, magnetic switch, photoelectric beam, among other types of sensors.

The prevention and detection of seizure events can include the use of filters to filter blue light of computing device displays and third-party computing devices capable of detecting seizure events and/or taking step to mitigate a seizure event. For example, a user may use a wearable sensor, such as a watch or headband, to detect a seizure event. The use of third-party devices to detect seizure events can be costly.

As such, the disclosure is directed to the detection of seizure events based on a monitored movement of a generated point cloud. For example, a computing device can include a non-transitory machine readable medium storing instructions executable by a processing resource to generate a point cloud of an object, monitor a movement of the generated point cloud based on data received from a sensor, and detect a seizure event based on the monitored movement. This may detect and/or mitigate seizure events in a less costly and less invasive way than other methods, such as wearable sensors.

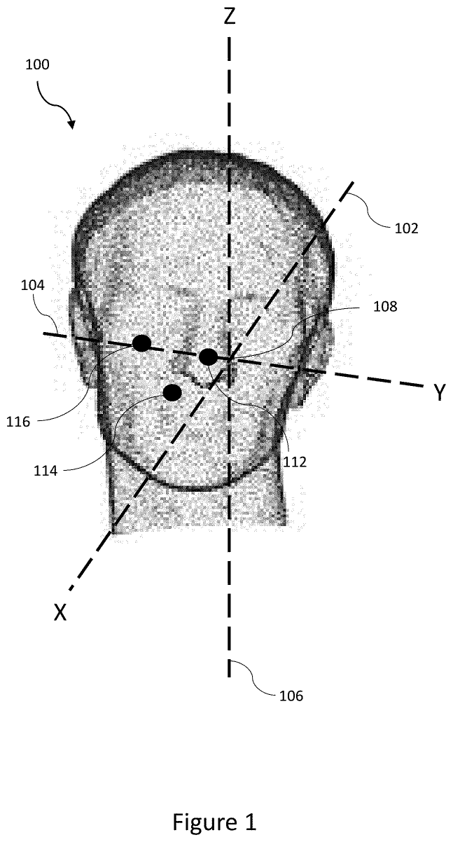

FIG. 1 is an example of a point cloud 100 for the detection of seizure events consistent with the disclosure. As used herein, the term “point cloud” refers to a collection of data points defined by a given coordinates system that represent an object. In a three-dimensional (3D) coordinates system, for example, a point cloud can be made up of a number of data points that may be identified by three coordinates that, taken together, correlate to a precise point in space relative to a point of origin.

As illustrated in FIG. 1, a x-axis 102, a y-axis 104, and a z-axis 106 may extend in two directions and the coordinates may identify a distance of the point from the intersection 108 of the x, y, z axes (0) and the direction of divergence, expressed as +or −. For example, the point cloud 100 may include a number of data points (e.g., 3D data points), such as data point 112, data point 114, and data point 116. Each of the number of data points may be identified by three coordinates that, taken together, correlate to a precise point in space relative to intersection 108. The first coordinate may be a coordinate along the x-axis 102, the second coordinate may be a coordinate along the y-axis 104, and the third coordinate may be a coordinate along the z-axis 106.

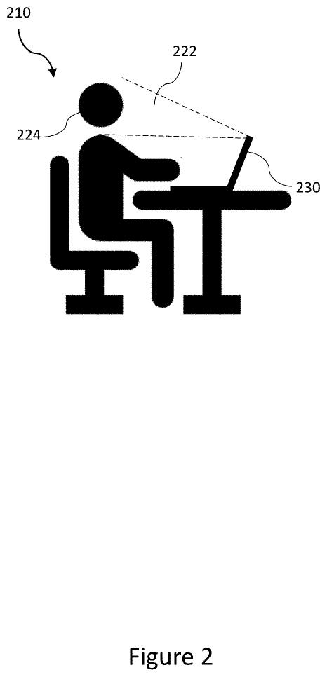

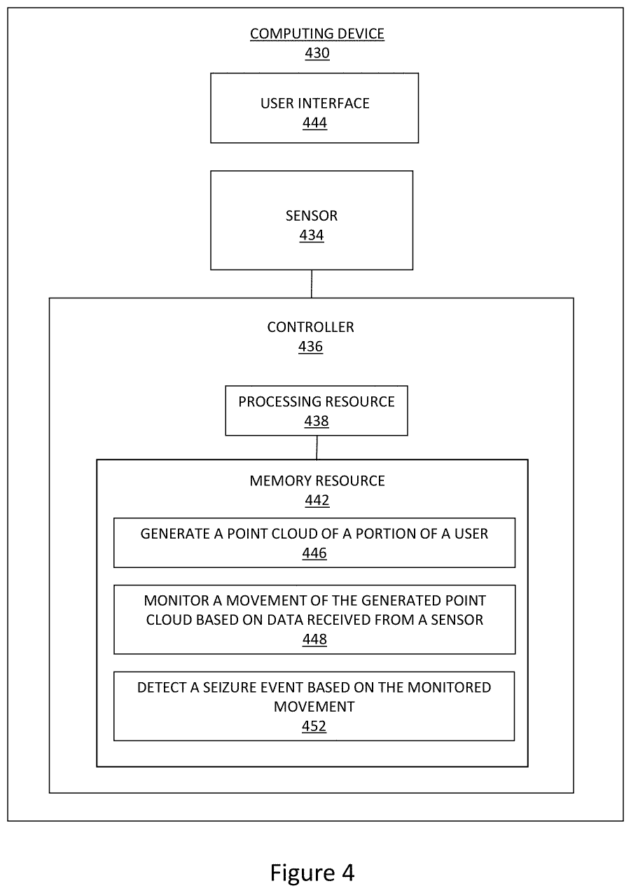

A computing device (e.g., computing device 430 illustrated in FIG. 4) may gather point measurements from real-world objects to generate the point cloud 100. For example, the computing device may gather point measurements of a user to generate the point cloud 100. In such an example, the generated point cloud 100 may represent a 3D model of a portion of a user, such as the user's head, that is positioned within a field of view (e.g., field of view 222 illustrated in FIG. 2) of a sensor (e.g., sensor 434 illustrated in FIG. 4) embedded within the computing device.

As described herein, a seizure event may be detected based on the movement of the generated point cloud 100. As used herein, the term “seizure event” refers to a sudden change in a user's movements. For example, a controller (e.g., controller 436 illustrated in FIG. 4) may monitor the movement of the generated point cloud 100 representing a user based on data received from the sensor. The data may include a number of data points of the generated point cloud 100, where each of the number of data points may include a 3D coordinate.

In some examples, the sensor may detect a movement of the generated point cloud 100 (e.g., movement of a user). In response to the detection of the movement, the sensor may send data to the controller to notify the controller of the movement. The controller may extract data points corresponding to a location of the generated point cloud 100 to monitor the movement of the generated point cloud 100. For example, the controller may identify a data point corresponding to a center point of the generated point cloud 100. In such an example, in response to the detection of movement of the generated point cloud 100, the controller can identify data points corresponding to the center point of the generated point cloud in real-time. There may be a series of data points corresponding to a center point of the generated point cloud 100 as the generated point cloud 100 moves. For example, the controller may extract a first data point 112 of the generated point cloud 100, which may correspond to the center point of the generated point cloud 100. As the generated point cloud 100 moves, the controller may extract additional data points that correspond to the center point of the generated point cloud 100.

For example, as illustrated in FIG. 1, there may be a first data point 112, a second data point 114, and a third data point 116. The first data point 112 may correspond to a first center point of the generated point cloud 100 when the generated point cloud 100 is in a first position. The second data point 114 may correspond to a second center point of the generated point cloud 100 when the generated point cloud 100 is in a second position. The third data point 116 may correspond to a third center point of the generated point cloud 100 when the generated point cloud 100 is in a third position. However, this disclosure is not so limited. Thus, the controller may monitor the movement of the number of data points of the generated point cloud 100 in relation to the intersection 108 of the x, y, z axes of the generated point cloud 100.

In some examples, the controller may identify a movement pattern of the extracted data points of the generated point cloud 100 as the generated point cloud 100 moves. For example, the controller may identify a movement pattern of the first data point 112, the second data point 114, and the third data point 116. The controller may compare the received data and/or the identified movement pattern of the received data points to seizure pattern data stored by the controller. As used herein, the term “seizure pattern data” refers to data associated with predictors of seizure events. For example, predictors of seizure events may be movement patterns that are indictive of a user experiencing a seizure, such as direction and/or speed of movements. The seizure pattern data may be stored by the controller. The controller may compare the received data to the stored seizure pattern data. For example, the identified movement pattern of extracted data points may be compared to the movement patterns that are indictive of a user experiencing a seizure. Based on the comparison of the received data to the seizure pattern data, the controller may detect a seizure event.

In response to the detection of a seizure event, a notification of the seizure event may be generated. The notification may include information associated with the seizure event, such as time, location, severity of the seizure event, etc. However, this disclosure is not so limited. The notification may be sent to a second computing device to notify a third party of the seizure event. For example, in response to the controller detecting a seizure event, the controller may send a notification to a mobile device to notify a third party. In some examples, in response to the detection of a seizure event, the controller may alter the operating state of the computing device. For example, the display of the computing device may be deactivated (e.g., turned off) in response to the detection of the seizure event.

As used herein, the term “display” is intended to mean hardware, circuitry, logic and a user interface, e.g., touch screen, to present content to a user. Thus, use of the term “display” herein is intended to include a user interface in the form of a screen capable of displaying information to a user. One example of a display includes a display having a light emitting diode (LED) screen type. Another example of a display may include a display having an organic light emitting diode (OLED) screen type. Examples, however, are not limited to these screen type technologies and other screen type technologies may be considered within the scope of this disclosure.

FIG. 2 is an example of a system 210 for the detection of seizure events consistent with the disclosure. The system 210 may include a computing device 230. While not illustrated in FIG. 2, the computing device 230 may include a sensor (e.g., sensor 434 illustrated in FIG. 4) and a controller (e.g., controller 436 illustrated in FIG. 4).

As described above, the computing device 230, via the controller, may generate a point cloud (e.g., point cloud 100 illustrated in FIG. 1) representing an object 224 within a point of view 222 of the sensor. For example, the generated point cloud may include a number of data points (e.g., 3D data points) within the point of view 222 of the sensor. The generated point cloud may be a 3D model of a portion of a user, such as the user's head, that is positioned within the field of view 222 of the sensor.

As described above, the sensor may detect a movement of the object 224. In response to the detection of movement of object 224, the sensor may send data associated with the generated point cloud of object 224 to the controller. The data may include a number of data points of the generated point cloud. Additionally, the controller may extract data points from the received number of data points that correspond to a center point of the generated point cloud, such as data point 112, data point 114, and data point 116 illustrated in FIG. 1. As described above, the data point corresponding to the center point of the generated point cloud may change as the generated point cloud moves. Thus, the controller may identify data points corresponding to the center point of the generated point cloud of object 224 in real-time to identify a movement pattern of the extracted data points.

In some examples, monitoring the movement of the generated point cloud may include monitoring the identified movement pattern of the extracted data points. The identified movement pattern of the identified data points may be compared to stored seizure pattern data that is stored within the controller. The controller may detect a seizure event in an instance where the identified movement pattern matches stored seizure pattern data.

In response to the detection of the seizure event, a notification of the seizure event may be sent to a computing device of a third party to notify the third party of the seizure event. Additionally, in response to the detection of the seizure event, the display of the computing device 230 may be deactivated (e.g., turned off).

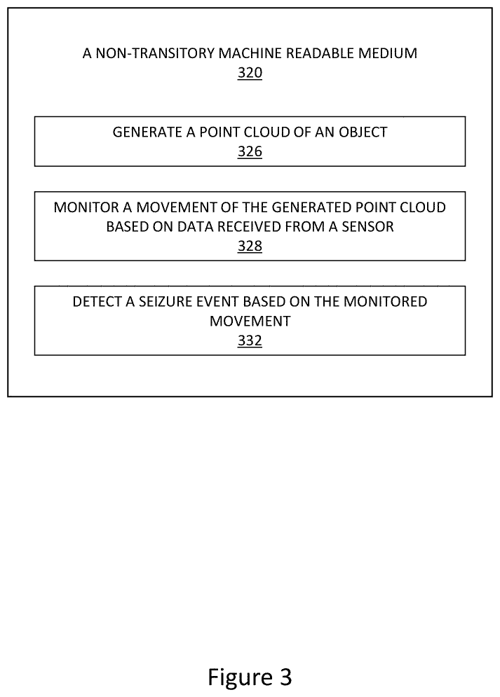

FIG. 3 illustrates an example of a memory resource 320 storing instructions 326, 328, 332 for the detection of seizure events consistent with the disclosure. In some examples, the memory resource 320 can be utilized to store instructions 326, 328, 332 that can be executed by a processing resource. For example, the memory resource 320 may be communicatively coupled to a processing resource which may be a central processing unit (CPU), a semiconductor-based microprocessor, and/or other hardware devices suitable for retrieval and execution of instructions 326, 328, 332 stored in the memory resource 320 (e.g., in a non-transitory computer readable medium). The example processing resource may fetch, decode, and execute instructions. As an alternative, or in addition to, retrieving and executing instructions, the example processor may include an electronic circuit that may include electronic components for performing the functionality of executed instructions.

In some examples, the processing resource may be a plurality of hardware processing units that may cause machine-readable instructions to be executed. The processing resource may include central processing units (CPUs) among other types of processing units. The memory resource 320 may be any type of volatile or non-volatile memory or storage, such as random-access memory (RAM), flash memory, storage volumes, a hard disk, or a combination thereof.

The memory resource 320 may store instructions thereon, such as instructions 326, 328, 332. When executed by the processing resource, the instructions may cause a computing device to perform specific tasks and/or functions. For example, the memory resource 320 may store instructions 326 which may be executed by the processing resource to cause the controller to generate a point cloud of an object. The controller may generate the point cloud of the object using a number of data points received from a sensor embedded within the computing device (e.g., computing device 230 illustrated in FIG. 2). The generated point cloud (e.g., generated point cloud 100 illustrated in FIG. 1) may be a 3D model of a portion of a user, such as the user's head, that is positioned within the field of view (e.g., point of view 222 illustrated in FIG. 2) of the sensor.

As illustrated in FIG. 3, the memory resource 320 may store instructions 328 which may be executed by the processing resource to cause the controller to monitor a movement of the generated point cloud based on data received from a sensor. For example, the controller may monitor the movement of data points corresponding to a center of the generated point cloud. In such an example, the controller may extract a number of data points corresponding to the center point of the generated point cloud as the generated point cloud moves.

As illustrated in FIG. 3, the memory resource 320 may store instructions 332 which may be executed by the processing resource to cause the controller to detect a seizure event based on the monitored movement. For example, the controller may monitor the movement of the extracted data points to identify a movement pattern of the generated point cloud. A movement pattern identified may be compared against and matched to seizure pattern data (e.g., a movement pattern identified as being indicative of a seizure event). When a match is identified, the controller may generate a notification of the detected seizure event, where the notification includes information associated with the seizure event, such as the time, location, and severity. However, this disclosure is not so limited.

In some examples, in response to the detection of the seizure event, the notification of the seizure event may be generated and sent to a third-party computing device to notify a third party of the seizure event. Additionally, in response to the detection of the seizure event, the display of the computing device may be deactivated to mitigate the seizure event.

FIG. 4 illustrates an example of a computing device 430 for the detection of seizure events consistent with the disclosure. The computing device 430 can include a sensor 434 and a controller 436. The computing device 430 can be, for example, computing device 230 described in connection with FIG. 2.

The controller 436 can include a processing resource 438 and a memory resource 442. The memory resource 442 can be any type of storage medium that can be accessed by the processing resource 438 to perform various examples of the present disclosure. For example, the memory resource 442 can be a non-transitory computer readable medium having computer readable instructions (e.g., computer program instructions) stored thereon that are executable by the processing resource 434 for detecting a seizure event based on a monitored movement of a generated point cloud. The generated point cloud can be, for example, generated point cloud 100, described in connection with FIG. 1.

The processing resource 438 of the controller 438 can execute instructions 446 to cause the controller 438 to generate a point cloud of an object. For example, the controller 438 can generate a point cloud of a portion of a user, such as the head of the user. The generated point cloud can include a set of data points within a point of view of a sensor, where the set of data points may represent the portion of the user that is positioned within the point of view of the sensor.

The memory resource 442 may store instructions 448 which may be executed by the processing resource 438 to monitor a movement of the generated point cloud based on data received from the sensor 434. As described above, the sensor 434 may detect a movement of the generated point cloud. For example, the sensor 434 may include a virtual accelerometer to monitor the movement of the generated point cloud in real-time. In response to detection of the movement of the generated point cloud, the sensor 434 may send data associated with the generated point cloud to the controller 436. The data may include a number of data points of the generated point cloud.

The controller 436 may extract data points from the received data that correspond to a center point of the generated point cloud. Additionally, the controller 436 may monitor the movement of the extracted data points in relation to an intersection (e.g., intersection 108 illustrated in FIG. 1) of a x, y, z axes of the generated point cloud to identify a movement pattern of the generated point cloud.

The memory resource 442 may store instructions 452 which may be executed by the processing resource 438 to detect a seizure event based on the monitored movement. For example, the seizure event may be detected based on a comparison of the data received from the sensor to stored seizure pattern data. In such an example, the controller may identify movement patterns of the extracted data points and compare the identified movement pattern to the stored seizure pattern data. The stored seizure pattern data may be movement patterns that are indicative of a user experiencing a seizure. Thus, if the identified movement pattern matches a stored seizure pattern, the controller 436 may detect a seizure event.

As described above, the controller 436 may include instructions which may be executed by the processing resource 438 to generate a notification of the seizure event. The generated notification may be sent to a second computing device to notify a third-party of the seizure event. In some examples, the display of the computing device may be deactivated in response to the detection of the seizure event.

As illustrated in FIG. 4, computing device 430 can include a user interface 444. A user can interact with computing device 430 via user interface 444. For example, user interface 444 can provide (e.g., display) information to and/or receive information from (e.g., input by) the user of computing device 430.

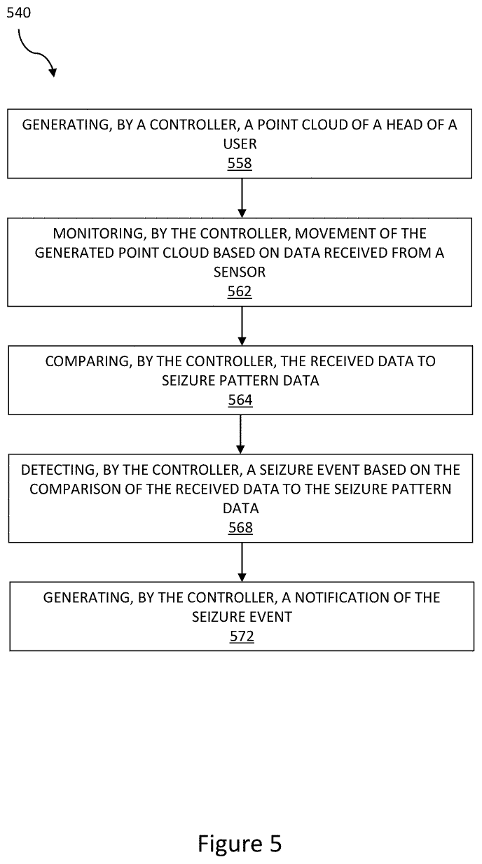

FIG. 5 illustrates an example of a method 540 for the detection of seizure events consistent with the disclosure. A seizure event can be detected using a controller and a sensor embedded within a first computing device, such as computing device 430 described in connection with FIG. 4. The controller can be, for example, controller 436 described in connection with FIG. 4. As described above, the controller can detect a seizure event based on a monitored movement of a generated point cloud.

At 558, the method 540 can include generating, by a controller, a point cloud of a head of a user. The point cloud of the head of a user can be generated by the controller, for example, in a manner analogous to that previously described in connection with FIGS. 1-4.

As described above, the generated point cloud (e.g., generated point cloud 100 illustrated in FIG. 1) may include a number of data points (e.g., 3D data points), such as data point 112, data point 114, and data point 116 illustrated in FIG. 1. Each of the number of data points may be identified by three coordinates that, taken together, correlate to a precise point in space relative to an intersection of an x, y, z axes of the generated point cloud. For example, the first coordinate may be a coordinate along the x-axis, the second coordinate may be a coordinate along the y-axis, and the third coordinate may be a coordinate along the z-axis.

At 562, the method 540 can include monitoring, by the controller, movement of the generated point cloud based on data received from a sensor. The movement of the generated point cloud can be monitored by the controller, for example, in a manner analogous to that previously described in connection with FIGS. 1-4. As described above, the movement of the generated point cloud can be monitored in response to a detection of movement by the sensor that is embedded within the first computing device.

As described above, the controller can extract number of data points from the data received from the sensor, where the number of extracted data points correspond to a center point of the generated point cloud. For example, a first data point may correspond to a first center point of the generated point cloud when the generated point cloud is in a first position. A second data point may correspond to a second center point of the generated point cloud when the generated point cloud is in a second position. A third data point may correspond to a third center point of the generated point cloud when the generated point cloud is in a third position. However, this disclosure is not so limited. Thus, the controller may monitor the movement of the number of extracted data points of the generated point cloud in relation to the intersection of the x, y, z axes of the generated point cloud to identify a movement pattern of the extracted data points.

At 564, the method 540 can include comparing, by the controller, the received data to seizure pattern data. The received data can be compared to seizure pattern data by the controller, for example, in a manner analogous to that previously described in connection with FIGS. 1-4. As described above, the controller may compare the identified movement pattern of the extracted data points to seizure pattern data stored by the controller. For example, the identified movement pattern of extracted data points may be compared to the movement patterns that are indictive of a user experiencing a seizure.

At 568, the method 540 can include detecting, by the controller, a seizure event based on the comparison of the received data to the seizure pattern data. The seizure event can be detected by the controller, for example, in a manner analogous to that previously described in connection with FIGS. 1-4. As described above, the controller may detect a seizure event in response to a determination that the identified movement pattern of the extracted data points matches a seizure pattern data.

At 572, the method 540 can include generating, by the controller, a notification of the seizure event. The notification of the seizure event can be generated by the controller, for example, in a manner analogous to that previously described in connection with FIGS. 1-4. As described above, the notification of the seizure event can also be sent to a second computing device to notify a third party of the seizure event. Additionally, the controller can deactivate a display of the first computing device in response to the detection of the seizure event to mitigate the seizure event.

In the foregoing detailed description of the disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electric, and/or structural changes may be made without departing from the scope of the disclosure. Further, as used herein, “a” can refer to one such thing or more than one such thing.

The figures herein follow a numbering convention in which the first digit corresponds to the drawing figure number and the remaining digits identify an element or component in the drawing. For example, reference numeral 102 may refer to element 102 in FIG. 1 and an analogous element may be identified by reference numeral 202 in FIG. 2. Elements shown in the various figures herein can be added, exchanged, and/or eliminated to provide additional examples of the disclosure. In addition, the proportion and the relative scale of the elements provided in the figures are intended to illustrate the examples of the disclosure, and should not be taken in a limiting sense.

It can be understood that when an element is referred to as being “on,” “connected to”, “coupled to”, or “coupled with” another element, it can be directly on, connected, or coupled with the other element or intervening elements may be present. In contrast, when an object is “directly coupled to” or “directly coupled with” another element it is understood that are no intervening elements (adhesives, screws, other elements) etc.

The above specification, examples and data provide a description of the method and applications, and use of the system and method of the disclosure. Since many examples can be made without departing from the spirit and scope of the system and method of the disclosure, this specification merely sets forth some of the many possible example configurations and implementations.

What is claimed is:

1. A non-transitory machine readable medium storing instructions executable by a processing resource to:
generate a point cloud of an object;
monitor a movement of the generated point cloud based on data received from a sensor; and
detect a seizure event based on the monitored movement.

2. The medium of claim 1, wherein the point cloud is a three-dimensional model of a head of a user, wherein the three-dimensional model includes a number of three-dimensional data points.

3. The medium of claim 1, further comprising instructions executable by the processing resource to:
store the data received from the sensor; and
store seizure pattern data, wherein the seizure pattern data is associated with identified movement patterns of seizure events.

4. The medium of claim 3, further comprising instructions executable by the processing resource to compare the data received from the sensor to the stored seizure pattern data to detect a seizure event.

5. The medium of claim 1, wherein the data includes a number of data points of the generated point cloud, wherein each of the number of data points include a three-dimensional coordinate.

6. The medium of claim 5, wherein the instructions to monitor movement of the generating point cloud includes instructions to monitor movement of one of the number of data points in relation to a three-dimensional axis of the generating point cloud.

7. The medium of claim 1, further comprising instructions executable by the processing resource to alter an operating state of a computing device of which the sensor is embedded within in response to detection of the seizure event.

8. A device, comprising:
a controller comprising instructions to:
generate a point cloud of a portion of a user;
monitor movement of the generated point cloud based on data received from a sensor; and
detect a seizure event based on the monitored movement; and
the sensor coupled to the controller to:
detect movement of the generated point cloud; and
send data corresponding to the detected movement to the controller.

9. The device of claim 8, wherein the generated point cloud includes a set of three-dimensional data points within a point of view of the sensor.

10. The device of claim 8, wherein the sensor includes a virtual accelerometer to monitor the movement of the generated point cloud in real-time.

11. The device of claim 8, further comprising the controller having instructions to extract a three-dimensional coordinate from the generated point cloud in response to the detection of movement of the generated point cloud, wherein the three-dimensional coordinate is a center point of the generated point cloud.

12. A method, comprising:
generating, by a controller, a point cloud of a head of a user;
monitoring, by the controller, movement of the generated point cloud based on data received from a sensor;
comparing, by the controller, the received data to seizure pattern data;
detecting, by the controller, a seizure event based on the comparison of the received data to the seizure pattern data; and
generating, by the controller, a notification of the seizure event.

13. The method of claim 12, wherein the movement of the generated point cloud is monitored in response to a detection of movement by a sensor that is embedded within a first computing device.

14. The method of claim 13, further comprising deactivating a display of the first computing device in response to the detection of the seizure event.

15. The method of claim 13, further comprising sending the notification of the seizure event to a second computing device to notify a third party of the seizure event.
